(19) United States
(12) Patent Application Publication
(10) Pub. No.: US 2015/0329914 A1
(43) Pub. Date: Nov. 19, 2015

(54) PREDICTIVE BIOMARKERS FOR PRE-MALIGNANT BREAST LESIONS

(71) Applicants: D. Joseph Jerry, Shutesbury, MA (US); Giovanna Crisi, Longmeadow, MA (US); Grace Makari-Judson, Suffield, CT (US); Karl Simin, Princeton, MA (US); Kelly J. Gregory, South Hadley, MA (US); Sallie Smith Schneider, Longmeadow, MA (US)

(72) Inventors: D. Joseph Jerry, Shutesbury, MA (US); Giovanna Crisi, Longmeadow, MA (US); Grace Makari-Judson, Suffield, CT (US); Karl Simin, Princeton, MA (US); Kelly J. Gregory, South Hadley, MA (US); Sallie Smith Schneider, Longmeadow, MA (US)

(21) Appl. No.: 14/707,886

(22) Filed: May 8, 2015

Related U.S. Application Data
(60) Provisional application No. 62/000,368, filed on May 19, 2014.

(51) Int. Cl. C12Q 1/68; G01N 33/574

(52) U.S. Cl. CPC C12Q 1/6886; G01N 33/57415; C12Q 2600/158; C12Q 2600/112; G01N 2333/4704; C12Q 2600/106

(57) ABSTRACT

The invention is based on the discovery of biomarkers and gene signatures that are useful for determining the presence of atypical hyperplasia in a breast lesion, and for determining whether a pre-malignant breast lesion is likely to progress to breast cancer. In particular, the present invention provides methods and reagents for detecting and profiling the expression levels of these biomarkers and genes, and methods of using the expression profiles for predicting the likelihood that a pre-malignant lesion will progress to breast cancer.

### CROSS-REFERENCE TO RELATED APPLICATION

This application claims priority to U.S. Provisional Application Ser. No. 62/000,368, filed on May 19, 2014, which is incorporated by reference herein in its entirety.

### FIELD OF THE INVENTION

The invention relates to biomarkers and gene signatures that are predictive of the risk that a pre-malignant lesion will progress to invasive carcinoma, as well as methods and assays for determining differential expression of the genes.

### BACKGROUND OF THE INVENTION

Breast cancer is the most common form of cancer in women and is second only to lung cancer as a cause of death. It is estimated that, based on current incidence rates, an American women has a one in eight chance of developing breast cancer at some time during her life. According to the American Cancer Society, in 2010, an estimated 207,090 new cases of invasive breast cancer were expected to be diagnosed in women in the U.S., along with 54,010 new cases of non-invasive (in situ) breast cancer.

As would be expected for such a major disease, the first efforts to apply emerging molecular and immunohistochemistry techniques in the 1980s to human cancers focused on breast cancer. Initial work considered the amplification of dormant oncogenes as prognostic markers and subsequently featured assessment of tumor suppressor genes. This was accompanied by a great interest in invasion and metastasis markers initially evaluated by immunohistochemistry and subsequently studied by molecular biologic techniques.

The goal of cancer prevention can be advanced by successful detection and treatment of pre-malignant non-invasive lesions. Pre-malignant breast lesions, also called atypical hyperplasia (AH), typically develop in ductal regions (ductal atypical hyperplasia) and lobular regions (lobular atypical hyperplasia) of breast tissue. Atypical hyperplasia is not a form of breast cancer; rather, it is a precursor in women who may be at risk for developing breast cancer in the future. Recent advances in breast cancer detection have made it possible to detect pre-malignant lesions; however, reproducibility in the diagnosis of atypical hyperplasias among pathologists is poor. The difficulty in diagnosis may contribute to an overdiagnosis such that fewer than 20% of pre-malignant breast lesions are obligate precursors of invasive carcinomas, making treatment of pre-malignant lesions of questionable value. Even when treated, up to 40% of patients undergoing prophylactic hormone therapies still develop invasive tumors. (1)

The success of mammography screening has increased the detection of pre-malignant lesions. However, overdiagnosis of breast cancer based on pre-malignant lesions has resulted in between about 50,000 to 70,000 woman annually being treated with surgery, radiation and hormonal therapies for lesions that, in all likelihood, would not have progressed to invasive cancer if left untreated. (2) Technological advances in imaging will only increase the scale of the challenge.

While there are a number of prognostic markers already in use for breast cancer to date, the need for powerful prognostic markers capable of identifying the subset of pre-malignant lesions that are most likely to advance to invasive cancer is apparent.

### SUMMARY OF THE INVENTION

The present invention provides objective biomarkers for the diagnosis of atypical hyperplasias, and gene expression profiles for predicting the risk that a pre-malignant breast lesion will advance to invasive breast cancer. The present invention further provides methods, assays and kits incorporating the present biomarkers and gene expression profiles.

In one aspect, the invention provides a single gene, SFRP 1, as a biomarker for the diagnosis of atypical hyperplasias (AH) in breast lesions. SFRP 1 is differentially expressed in pre-malignant lesions, compared to its expression in undiseased tissue and benign lesions. In one embodiment, the invention provides a method for diagnosing AH in a breast lesion, the method comprising obtaining a tissue sample from the patient by excision, aspiration or biopsy, assaying the sample by one or more colorimetric methods to determine the level of expression of SFRP1, and comparing the expression level of SFRP1 in the lesion with the level of expression of SFRP1 in normal breast tissue (i.e., breast tissue that is free of AH). The presence of AH is confirmed if the level of SFRP1 is down-regulated (under-expressed) compared to its expression level in the normal breast tissue.

The invention further provides a method for diagnosing the presence of AH in a pre-malignant breast lesion, the method comprising obtaining a tissue sample from the patient by excision, aspiration or biopsy, assaying the sample by one or more colorimetric methods to determine the level of expression of, and comparing the expression level of SFRP1 in the pre-malignant lesion with the level of expression of in normal breast tissue (i.e., breast tissue that is free of AH). The presence of AH is confirmed if the level of SFRP1 is down-regulated (under-expressed) compared to its expression level in the normal breast tissue.

In another aspect, the invention provides a gene signature for predicting whether a pre-malignant lesion will advance to invasive cancer. The gene signature contains between about 7 and about 33 genes that are differentially expressed in pre-malignant lesions that are highly likely to advance to invasive breast concern one embodiment, the gene signature includes all or a subcombination of the following 33 genes: TP53RK, GLUL, MKNK2, KDM4B, NAPA, TTC39A, GREB1, POTEE, POTEM, TMEM25, DNALI1, MLPH, FOXA1, PREX1, KIAA1244, AR, CACNA1D, ESR1, LRIG1, CRYAB, MAML2, DMD, TFAP2C, SFRP1, NFIB, ARRDC3, ANXA1, CXCL2, SLP1, MSX2, PRKAR2B, SGK (1 and/or 3) and ANKRD36

In an embodiment, the gene signature includes all or a subcombination of the following 8 genes: TP53RK, KDM4B, GREB1, FOXA1, ESR1, MAML2, SFRP1 and ANXA1. In an aspect, TP53RK, KDM4B, GREB1, FOXA1 and ESR1 are up-regulated (over-expressed), and MAML2, SFRP1 and ANXA1 are down-regulated (under-expressed) in pre-malignant lesions that are highly likely to advance to invasive breast cancer.

The invention further provides a method for predicting a clinical outcome of a patient diagnosed with a pre-malignant breast lesion, the method comprising obtaining a tissue sample from the patient by excision, aspiration or biopsy, assaying the sample by one or more colorimetric methods to determine the level of expression of all or a subcombination of the following 33 genes: TP53RK, GLUL, MKNK2, KDM4B, NAPA, TTC39A, GREB1, POTEE, POTEM, TMEM25, DNALI1, MLPH, FOXA1, PREX1, KIAA1244, AR, CACNA1D, ESR1, LRIG1, CRYAB, MAML2, DMD, TFAP2C, SFRP1, NFIB, ARRDC3, ANXA1, CXCL2, SLP1, MSX2, PRKAR2B, SGK (1 and/or 3) and ANKRD36.

In an embodiment, the gene signature includes all or a subcombination of the following 8 genes: TP53RK, KDM4B, GREB1, FOXA1, ESR1, MAML2, SFRP1 and ANXA1, and comparing the expression levels of all or a subcombination of TP53RK, KDM4B, GREB1, FOXA1, ESR1, MAML2, SFRP1 and ANXA1 in the pre-malignant lesion with the levels of expression of these genes in normal breast tissue (i.e., breast tissue that is free of AH). If the level of TP53RK, KDM4B, GREB1, FOXA1 or ESR1 are up-regulated, and/or the level of MAML2, SFRP1 or ANXA1 are down-regulated in the pre-malignant lesions compared to their expression levels in the normal breast tissue, then the pre-malignant lesion is likely to advance to invasive carcinoma.

In one embodiment the sample from the patient is selected from the group consisting of epithelial cells or tissue, ductal components, lymph fluid and inflammatory cells or combinations of these.

Assays of the present invention include immunoassays. These may include any immunoassay format, including but not limited to ELISAs, IHC or other colorimetric assay.

### DETAILED DESCRIPTION OF THE INVENTION

The biomarkers and gene signatures provided here represent an improvement over mere morphologic parameter or feature assessments of pre-malignant lesions. A range of lesions has been identified that are associated with increased breast cancer risk; however, the morphological assessments used presently are subject to poor reproducibility, making the diagnosis of premalignant lesions uncertain (4). There are approximately 50,000 diagnoses of pre-malignant breast lesions annually in the United States, with about 20% expected to progress to invasive breast cancers (5). This represents 10,000 women each year for whom breast cancer could be prevented if tools for accurate diagnosis of these high risk lesions and appropriate treatments were available.

At present, hormone blocking therapies are the primary treatment offered to women for reducing the risk of subsequent breast cancer; hormone therapy has been shown to reduce progression to invasive cancer by 56% for lobular neoplasia (lobular carcinoma in situ) and 86% for atypical ductal hyperplasia (1). However, the treatment fails to protect significant numbers of women, suggesting that endocrine targeted therapies are not appropriate in all cases. For women bearing underlying deficiencies in DNA repair pathways that render them susceptible (6), additional therapies may be necessary. Genetic background also can modify a patient's susceptibility to tumors. (3;7). Similarly, differences in genetic background among women may explain the relatively poor clinical utility of somatic mutations in TP53 in predicting breast cancer outcomes. Therefore, it is critical to develop objective diagnostic tools to reproducibly distinguish pre-malignant lesions, and which can identify the subset of lesions that are at high risk of progression.

### Definitions

Unless otherwise defined, all technical and scientific terms used herein have the same meaning as commonly understood by one of ordinary skill in the art to which this invention belongs. Although methods and materials similar or equivalent to those described herein can be used in the practice or testing of methods featured in the invention, suitable methods and materials are described below.

For convenience, the meaning of certain terms and phrases employed in the specification, examples, and appended claims are provided below. The definitions are not meant to be limiting in nature and serve to provide a clearer understanding of certain aspects of the present invention.

The term “hyperplasia” means an abnormality in a cell's appearance in which there are more cells than would be expected in a certain location, e.g., the walls of the ducts or lobules, but that all of these cells appear normal. The term “atypia” or “atypical” means that the cells look different from normal cells, but that they don't have all the features of cancer cells. Atypia may occur with hyperplasia (“atypical hyperplasia” or “AH”), which means that the cells look different from normal, and that there are more cells than would be expected in the location. Atypia also may occur in breast tissue without having hyperplasia.

The terms “ductal” and “lobular” indicate where cells originate within the breast. Ductal means that the cells are in the ducts, the passages that the milk travels through to get to the nipple. Lobular means that the cells are in the lobules, the parts of the breast capable of making milk. “Ductal Atypical Hyperplasia” or “DAH” refers to AH located in ductal tissues; “Lobular Atypical Hyperplasia” or “LAH” refers to AH located in lobular tissues;

The term “genome” is intended to include the entire DNA complement of an organism, including the nuclear DNA component, chromosomal or extrachromosomal DNA, as well as the cytoplasmic domain (e.g., mitochondrial DNA).

The term “gene” refers to a nucleic acid sequence that comprises control and most often coding sequences necessary for producing a polypeptide or precursor. Genes, however, may not be translated and instead code for regulatory or structural RNA molecules.

A gene may be derived in whole or in part from any source known to the art, including a plant, a fungus, an animal, a bacterial genome or episome, eukaryotic, nuclear or plasmid DNA, cDNA, viral DNA, or chemically synthesized DNA. A gene may contain one or more modifications in either the coding or the untranslated regions that could affect the biological activity or the chemical structure of the expression product, the rate of expression, or the manner of expression control. Such modifications include, but are not limited to, mutations, insertions, deletions, and substitutions of one or more nucleotides. The gene may constitute an uninterrupted coding sequence or it may include one or more introns, bound by the appropriate splice junctions.

The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

The phrase “single-gene marker” or “single gene marker” refers to a single gene (including all variants of the gene) expressed by a particular cell or tissue type wherein presence of the gene or transcriptional products thereof, taken individually the differential expression of such, is indicative/predictive of a certain condition.

The term “nucleic acid” as used herein, refers to a molecule comprised of one or more nucleotides, i.e., ribonucleotides, deoxyribonucleotides, or both. The term includes monomers and polymers of ribonucleotides and deoxyribonucleotides, with the ribonucleotides and/or deoxyribonucleotides being bound together, in the case of the polymers, via 5′ to 3′ linkages. The ribonucleotide and deoxyribonucleotide polymers may be single or double-stranded. However, linkages may include any of the linkages known in the art including, for example, nucleic acids comprising 5′ to 3′ linkages. The nucleotides may be naturally occurring or may be synthetically produced analogs that are capable of forming base-pair relationships with naturally occurring base pairs. Examples of non-naturally occurring bases that are capable of forming base-pairing relationships include, but are not limited to, aza and deaza pyrimidine analogs, aza and deaza purine analogs, and other heterocyclic base analogs, wherein one or more of the carbon and nitrogen atoms of the pyrimidine rings have been substituted by heteroatoms, e.g., oxygen, sulfur, selenium, phosphorus, and the like.

The term “complementary” as it relates to nucleic acids refers to hybridization or base pairing between nucleotides or nucleic acids, such as, for example, between the two strands of a double-stranded DNA molecule or between an oligonucleotide probe and a target are complementary.

As used herein, an “expression product” is a biomolecule, such as a protein or mRNA, which is produced when a gene in an organism is expressed. An expression product may comprise post-translational modifications. The polypeptide of a gene may be encoded by a full length coding sequence or by any portion of the coding sequence.

The term “derivative” is used synonymously with the term “variant” and refers to a molecule that has been modified or changed in any way relative to a reference molecule or starting molecule.

The terms “array” and “microarray” refer to any type of regular arrangement of objects usually in rows and columns. As it relates to the study of gene and/or protein expression, arrays refer to an arrangement of probes (often oligonucleotide or protein based) or capture agents anchored to a surface which are used to capture or bind to a target of interest. Targets of interest may be genes, products of gene expression, and the like. The type of probe (nucleic acid or protein) represented on the array is dependent on the intended purpose of the array (e.g., to monitor expression of human genes or proteins). The oligonucleotide- or protein-capture agents on a given array may all belong to the same type, category, or group of genes or proteins. Genes or proteins may be considered to be of the same type if they share some common characteristics such as species of origin (e.g., human, mouse, rat); disease state (e.g., cancer); structure or functions (e.g., protein kinases, tumor suppressors); or same biological process (e.g., apoptosis, signal transduction, cell cycle regulation, proliferation, differentiation). For example, one array type may be a “cancer array” in which each of the array oligonucleotide- or protein-capture agents correspond to a gene or protein associated with a cancer. An “epithelial array” may be an array of oligonucleotide- or protein-capture agents corresponding to unique epithelial genes or proteins. Similarly, a “cell cycle array” may be an array type in which the oligonucleotide- or protein-capture agents correspond to unique genes or proteins associated with the cell cycle.

The terms “immunohistochemical” or as abbreviated “IHC” as used herein refer to the process of detecting antigens (e.g., proteins) in a biologic sample by exploiting the binding properties of antibodies to antigens in said biologic sample.

The term “immunoassay” refers to a test that uses the binding of antibodies to antigens to identify and measure certain substances. Immunoassays often are used to diagnose disease, and test results can provide information about a disease that may help in planning treatment. An immunoassay takes advantage of the specific binding of an antibody to its antigen. Monoclonal antibodies are often used as they usually bind only to one site of a particular molecule, and therefore provide a more specific and accurate test, which is less easily confused by the presence of other molecules. The antibodies used must have a high affinity for the antigen of interest, because a very high proportion of the antigen must bind to the antibody in order to ensure that the assay has adequate sensitivity.

The term “PCR” or “RT-PCR”, abbreviations for polymerase chain reaction technologies, as used here refer to techniques for the detection or determination of nucleic acid levels, whether synthetic or expressed.

The term “cell type” refers to a cell from a given source (e.g., a tissue, organ) or a cell in a given state of differentiation, or a cell associated with a given pathology or genetic makeup.

The term “activation” as used herein refers to any alteration of a signaling pathway or biological response including, for example, increases above basal levels, restoration to basal levels from an inhibited state, and stimulation of the pathway above basal levels.

The term “differential expression” refers to both quantitative as well as qualitative differences in the temporal and tissue expression patterns of a gene or a protein in diseased tissues or cells versus normal adjacent tissue. For example, a differentially expressed gene may have its expression activated or completely inactivated in normal versus disease conditions, or may be up-regulated (over-expressed) or down-regulated (under-expressed) in a disease condition versus a normal condition. Such a qualitatively regulated gene may exhibit an expression pattern within a given tissue or cell type that is detectable in either control or disease conditions, but is not detectable in both. Stated another way, a gene or protein is differentially expressed when expression of the gene or protein occurs at a higher or lower level in the diseased tissues or cells of a patient relative to the level of its expression in the normal (disease-free) tissues or cells of the patient and/or control tissues or cells. Significant differential expression typically is at least about 2-fold over- or under-expression compared to normal disease free tissue.

The term “detectable” refers to an RNA expression pattern which is detectable via the standard techniques of polymerase chain reaction (PCR), reverse transcriptase-(RT) PCR, differential display, and Northern analyses, or any method which is well known to those of skill in the art. Similarly, protein expression patterns may be “detected” via standard techniques such as Western blots.

The term “complementary” as it relates to arrays refers to the topological compatibility or matching together of the interacting surfaces of a probe molecule and its target. The target and its probe can be described as complementary, and furthermore, the contact surface characteristics are complementary to each other.

By “amplification” is meant production of multiple copies of a target nucleic acid that contains at least a portion of an intended specific target nucleic acid sequence. The multiple copies may be referred to as amplicons or amplification products. In one embodiment, the amplified target contains less than the complete target gene sequence (introns and exons) or an expressed target gene sequence (spliced transcript of exons and flanking untranslated sequences). For example, FAS-specific amplicons may be produced by amplifying a portion of the FAS target polynucleotide by using amplification primers which hybridize to, and initiate polymerization from, internal positions of the FAS target polynucleotide. In another embodiment, the amplified portion contains a detectable target sequence which may be detected using any of a variety of well known methods.

By “primer” or “amplification primer” is meant an oligonucleotide capable of binding to a region of a target nucleic acid or its complement and promoting nucleic acid amplification of the target nucleic acid. In most cases a primer will have a free 3′ end which can be extended by a nucleic acid polymerase. All amplification primers include a base sequence capable of hybridizing via complementary base interactions either directly with at least one strand of the target nucleic acid or with a strand that is complementary to the target sequence. Amplification primers serve as substrates for enzymatic activity that produces a longer nucleic acid product.

A “target-binding sequence” of an amplification primer is the portion that determines target specificity because that portion is capable of annealing to a target nucleic acid strand or its complementary strand. The complementary target sequence to which the target-binding sequence hybridizes is referred to as a primer-binding sequence.

By “detecting” an amplification product is meant any of a variety of methods for determining the presence of an amplified nucleic acid, such as, for example, hybridizing a labeled probe to a portion of the amplified product. A labeled probe is an oligonucleotide that specifically binds to another sequence and contains a detectable group which maybe, for example, a fluorescent moiety, a chemiluminescent moiety, a radioisotope, biotin, avidin, enzyme, enzyme substrate, or other reactive group.

By “nucleic acid amplification conditions” is meant environmental conditions including salt concentration, temperature, the presence or absence of temperature cycling, the presence of a nucleic acid polymerase, nucleoside triphosphates, and cofactors which are sufficient to permit the production of multiple copies of a target nucleic acid or its complementary strand using a nucleic acid amplification method. Many well-known methods of nucleic acid amplification require thermocycling to alternately denature double-stranded nucleic acids and hybridize primers.

The term “biomarker” as used herein refers to a characteristic that is objectively measured and evaluated as an indicator of normal biological processes, pathogenic processes or biological responses to a therapeutic intervention. They can suggest etiology of, susceptibility to, activity of or progress of a disease substance indicative of a biological state.

The term “biological sample” or “biologic sample” refers to a sample obtained from an organism (e.g., a human patient) or from components (e.g., tissue, cells) or from body fluids (e.g., blood, serum, sputum, urine, etc) of an organism. The sample may be of any biological tissue, organ, organ system or fluid. The sample may be a “clinical sample” which is a sample derived from a patient. Such samples include, but are not limited to, sputum, blood, blood cells (e.g., white cells), plasma, bone marrow, and tissue or core, fine or punch needle biopsy samples, aspirations, urine, or cells therefrom. Biological samples may also include sections of tissues such as frozen sections taken for histological purposes. A biological sample may also be referred to as a “patient sample.”

The term “condition” refers to the status of any cell, organ, organ system or organism. Conditions may reflect a disease state or simply the physiologic presentation or situation of an entity. Conditions may be characterized as phenotypic conditions such as the macroscopic presentation of a disease or genotypic conditions such as the underlying gene or protein expression profiles associated with the condition. Conditions may be benign or malignant.

The term “cancer” in an individual refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an individual, or may circulate in the blood stream as independent cells, such as leukemic cells.

The term “breast cancer” means a cancer of the breast tissue or associated lymph nodes.

The term “cell growth” is principally associated with growth in cell numbers, which occurs by means of cell reproduction (i.e. proliferation) when the rate of the latter is greater than the rate of cell death (e.g. by apoptosis or necrosis), to produce an increase in the size of a population of cells, although a small component of that growth may in certain circumstances be due also to an increase in cell size or cytoplasmic volume of individual cells. An agent that inhibits cell growth can thus do so by either inhibiting proliferation or stimulating cell death, or both, such that the equilibrium between these two opposing processes is altered.

The term “tumor growth” or “tumor metastases growth”, as used herein, unless otherwise indicated, is used as commonly used in oncology, where the term is principally associated with an increased mass or volume of the tumor or tumor metastases, primarily as a result of tumor cell growth.

The term “metastasis” means the process by which cancer spreads from the place at which it first arose as a primary tumor to distant locations in the body. Metastasis also refers to cancers resulting from the spread of the primary tumor. For example, someone with breast cancer may show metastases in their lymph system, liver, bones or lungs.

The term “lesion” or “lesion site” as used herein refers to any abnormal, generally localized, structural change in a bodily part or tissue. Calcifications or fibrocystic features are examples of lesions of the present invention.

A “pre-malignant lesion” refers to an abnormal condition or lesion that is not actively growing (i.e., malignant) but that typically precedes or develops into a malignancy. AH, including LAH and DAH, are considered to be pre-malignant lesions.

The term “clinical management parameter” refers to a metric or variable considered important in the detecting, screening, diagnosing, staging or stratifying patients, or determining the progression of, regression of and/or survival from a disease or condition. Examples of such clinical management parameters include, but are not limited to survival in years, disease related death, early or late recurrence, degree of regression, metastasis, responsiveness to treatment, effectiveness of treatment or the likelihood of progression to breast cancer.

The term “endpoint” means a final stage or occurrence along a path or progression or discrete measurement (e.g. level of expression of a gene).

The phrase “morphologic prognosis parameter or feature” means a feature of the cancerous phenotype used to predict an outcome. Morphologic prognosis parameters or features include pre-malignant lesions (including AH), axillary lymph node metastasis, tumor type, tumor grade, and tumor size. Secondary but important morphologic parameters also considered predictive include the extent of an intraductal component in patients with mixed intraductal and infiltrating ductal carcinoma, proven intralymphatic and intravascular invasion, and high mitotic index.

The phrase “lymph node negative” as used herein refers to the status of a patient where at least one or more removed or biopsied lymph nodes showed no evidence of metastatic carcinoma. In one embodiment, a lymph node negative status is defined as the situation where more than 4, more than 5 or more than 6 removed or biopsied lymph nodes showed no evidence of metastatic carcinoma.

The term “treating” as used herein, unless otherwise indicated, means reversing, alleviating, inhibiting the progress of, or preventing, either partially or completely, the growth of tumors, tumor metastases, or other cancer-causing or neoplastic cells in a patient with cancer. The term “treatment” as used herein, unless otherwise indicated, refers to the act of treating.

The phrase “a method of treating” or its equivalent, when applied to, for example, cancer refers to a procedure or course of action that is designed to reduce, eliminate or prevent the number of cancer cells in an individual, or to alleviate the symptoms of a cancer. “A method of treating” cancer or another proliferative disorder does not necessarily mean that the cancer cells or other disorder will, in fact, be completely eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of an individual, is nevertheless deemed an overall beneficial course of action.

The term “predicting” or “predict” means a statement or claim that a particular event will, or is very likely to, occur in the future.

The term “prognosing” or “prognosis” means a statement or claim that a particular biologic event will, or is very likely to, occur in the future.

The term “progression” or “cancer progression” means the advancement or worsening of or toward a disease or condition.

The term “therapeutically effective agent” means a composition that will elicit the biological or medical response of a tissue, organ, system, organism, animal or human that is being sought by the researcher, veterinarian, medical doctor or other clinician.

The term “therapeutically effective amount” or “effective amount” means the amount of the subject compound or combination that will elicit the biological or medical response of a tissue, organ, system, organism, animal or human that is being sought by the researcher, veterinarian, medical doctor or other clinician.

The term “correlate” or “correlation” as used herein refers to a relationship between two or more random variables or observed data values. A correlation may be statistical if, upon analysis by statistical means or tests, the relationship is found to satisfy the threshold of significance of the statistical test used.

one marker was tested.

### Gene Signatures

The present invention provides biomarkers, including gene signatures, for accurately identifying high-risk pre-malignant lesions, i.e., those that are likely to advance to breast cancer. The invention provides superior assays and methods, involving measuring the expression level of at least one of the following genes: TP53RK, GLUL, MKNK2, KDM4B, NAPA, TTC39A, GREB1, POTEE, POTEM, TMEM25, DNALI1, MLPH, FOXA1, PREX1, KIAA1244, AR, CACNA1D, ESR1, LRIG1, CRYAB, MAML2, DMD, TFAP2C, SFRP1, NFIB, ARRDC3, ANXA1, CXCL2, SLP1, MSX2, PRKAR2B, SGK (1 and/or 3) and ANKRD36.

In one aspect, the invention provides all or a subcombination of a 33-gene signature for predicting whether a pre-malignant lesion will advance to invasive cancer comprising the following genes: TP53RK, GLUL, MKNK2, KDM4B, NAPA, TTC39A, GREB1, POTEE, POTEM, TMEM25, DNALI1, MLPH, FOXA1, PREX1, KIAA1244, AR, CACNA1D, ESR1, LRIG1, CRYAB, MAML2, DMD, TFAP2C, SFRP1, NFIB, ARRDC3, ANXA1, CXCL2, SLP1, MSX2, PRKAR2B, SGK (1 and/or 3) and ANKRD36. The genes comprising the present gene signature are differentially expressed in pre-malignant lesions that are highly likely to advance to invasive breast cancer.

In an embodiment, the gene signature includes all or a subcombination of the following 8 genes: TP53RK, KDM4B, GREB1, FOXA1, ESR1, MAML2, SFRP1 and ANXA1. In one aspect, TP53RK, KDM4B, GREB1, FOXA1 and ESR1 are up-regulated (over-expressed), and MAML2, SFRP1 and ANXA1 are down-regulated (under-expressed) in pre-malignant lesions that are highly likely to advance to invasive breast cancer.

The invention further provides a method for predicting a clinical outcome of a patient diagnosed with a pre-malignant breast lesion, the method comprising obtaining a tissue sample from the patient by excision, aspiration or biopsy, assaying the sample by one or more colorimetric methods to determine the level of expression of all or a subcombination of the following 8 genes: TP53RK, KDM4B, GREB1, FOXA1, ESR1, MAML2, SFRP1 and ANXA1, and comparing the expression levels of all or a subcombination of TP53RK, KDM4B, GREB1, FOXA1, ESR1, MAML2, SFRP1 and ANXA1 in the pre-malignant lesion with the levels of expression of these genes in normal breast tissue (i.e., breast tissue that is free of AH). If the level of TP53RK, KDM4B, GREB1, FOXA1 or ESR1 are up-regulated, and/or the level of MAML2, SFRP1 or ANXA1 are down-regulated in the pre-malignant lesions compared to their expression levels in the normal breast tissue, then the pre-malignant lesion is likely to advance to invasive carcinoma.

In another aspect, the invention provides a single gene, SFRP 1, as a biomarker for the diagnosis of atypical hyperplasias (AH) in breast lesions. SFRP 1 is differentially expressed in pre-malignant lesions, compared to its expression in non-diseased tissue and benign lesions. In one embodiment, the invention provides a method for diagnosing AH in a breast lesion, the method comprising obtaining a tissue sample from the patient by excision, aspiration or biopsy, assaying the sample by one or more colorimetric methods to determine the level of expression of SFRP1, and comparing the expression level of SFRP1 in the lesion with the level of expression of SFRP1 in normal breast tissue (i.e., breast tissue that is free of AH). The presence of AH is confirmed if the level of SFRP1 is down-regulated (under-expressed) compared to its expression level in the normal breast tissue.

The invention further provides a method for diagnosing the presence of AH in a pre-malignant breast lesion, the method comprising obtaining a tissue sample from the patient by excision, aspiration or biopsy, assaying the sample by one or more colorimetric methods to determine the level of expression of, and comparing the expression level of SFRP1 in the pre-malignant lesion with the level of expression of in normal breast tissue (i.e., breast tissue that is free of AH). The presence of AH is confirmed if the level of SFRP1 is down-regulated (under-expressed) compared to its expression level in the normal breast tissue.

The sample from the patient may be selected from the group consisting of epithelial cells or tissue, ductal components, lymph fluid and inflammatory cells or combinations of these.

In an embodiment, the present invention comprises methods for determining gene expression profiles that are indicative of the likelihood that a pre-malignant lesion will advance to breast cancer. The present method comprises (a) obtaining a biological sample (e.g., tissue biopsy of the lesion site) of a patient diagnosed as having a breast lesion; (b) contacting the sample with nucleic acid probes specific for all or a subcombination of the following genes: TP53RK, GLUL, MKNK2, KDM4B, NAPA, TTC39A, GREB1, POTEE, POTEM, TMEM25, DNALI1, MLPH, FOXA1, PREX1, KIAA1244, AR, CACNA1D, ESR1, LRIG1, CRYAB, MAML2, DMD, TFAP2C, SFRP1, NFIB, ARRDC3, ANXA1, CXCL2, SLP1, MSX2, PRKAR2B, SGK (1 and/or 3) and ANKRD36; and (c) determining whether one or more of these genes are up-regulated (over-expressed) or down-regulated. The predictive value of the gene profile for determining the likelihood of progression to cancer increases with the number of these genes that are found to be up- or down-regulated in accordance with the invention. In one embodiment, at least about two, such as at least about four, or least about eight, of the genes in the present GPEP are differentially expressed. The biological sample can be a sample of the patient's primary lesion; normal (undiseased) marginal breast tissue from the same patient is used as a control. In one embodiment, expression of at least two reference genes also is measured.

In another embodiment of the method, the gene expression profile comprises measuring all or a subcombination of the following 8 genes: TP53RK, KDM4B, GREB1, FOXA1, ESR1, MAML2, SFRP1 and ANXA1, and comparing the expression levels of all or a subcombination of these genes in the pre-malignant lesion with the levels of expression of these genes in normal breast tissue (i.e., breast tissue that is free of AH). If the level of TP53RK, KDM4B, GREB1, FOXA1 and/or ESR1 are up-regulated, and/or the level of MAML2, SFRP1 and/or ANXA1 are down-regulated in the pre-malignant lesions compared to their expression levels in the normal breast tissue, then the pre-malignant lesion is likely to advance to invasive carcinoma.

In another aspect, the method comprises measuring the expression level of a single gene, SFRP1, as a biomarker for diagnosing the presence of AH in a breast lesion. SFRP1 is differentially expressed in pre-malignant lesions, compared to its expression in non-diseased tissue and benign lesions. Specifically, the presence of AH is confirmed if the level of SFRP 1 is down-regulated (under-expressed) compared to its expression level in the normal breast tissue.

In an alternative embodiment of the invention, the expression of proteins in a biological sample from a patient having a pre-malignant breast lesion is assayed using immunohistochemistry or immunoassay techniques to identify the expression of proteins in the present GPEP. In one embodiment, the protein expression profile comprises all or a subcombination of proteins encoded by the following genes: TP53RK, GLUL, MKNK2, KDM4B, NAPA, TTC39A, GREB1, POTEE, POTEM, TMEM25, DNALI1, MLPH, FOXA1, PREX1, KIAA1244, AR, CACNA1D, ESR1, LRIG1, CRYAB, MAML2, DMD, TFAP2C, SFRP1, NFIB, ARRDC3, ANXA1, CXCL2, SLP1, MSX2, PRKAR2B, SGK (1 and/or 3) and ANKRD36. According to the invention, some or all of these proteins are differentially expressed in patients having pre-malignant lesions that are at risk for progressing to cancer.

In this embodiment, the method comprises (a) obtaining a biological sample of a patient identified as having a pre-malignant breast lesion; (b) contacting the sample with nucleic acid probes or antibodies specific for the proteins encoded by all or a subcombination of the following genes: TP53RK, GLUL, MKNK2, KDM4B, NAPA, TTC39A, GREB1, POTEE, POTEM, TMEM25, DNALI1, MLPH, FOXA1, PREX1, KIAA1244, AR, CACNA1D, ESR1, LRIG1, CRYAB, MAML2, DMD, TFAP2C, SFRP1, NFIB, ARRDC3, ANXA1, CXCL2, SLP1, MSX2, PRKAR2B, SGK (1 and/or 3) and ANKRD36; and (c) determining whether one or more of these proteins are up-regulated (over-expressed) or down-regulated (under-expressed) in the pre-malignant lesion compared to their expression levels in normal tissue.

In one embodiment of the method, the protein expression profile comprises measuring all or a subcombination of the proteins encoded by the following 8 genes: TP53RK, KDM4B, GREB1, FOXA1, ESR1, MAML2, SFRP1 and ANXA1, and comparing the expression levels of all or a subcombination of these proteins in the pre-malignant lesion with the levels of expression of these proteins in normal breast tissue (i.e., breast tissue that is free of AH). If the level of TP53RK, KDM4B, GREB1, FOXA1 and/or ESR1 are up-regulated, and/or the level of MAML2, SFRP1 and/or ANXA1 are down-regulated in the pre-malignant lesions compared to their expression levels in the normal breast tissue, then the pre-malignant lesion is likely to advance to invasive carcinoma.

In another aspect, the method comprises a method for diagnosing the presence of AH in a pre-malignant breast lesion, the method comprising obtaining a tissue sample from the patient by excision, aspiration or biopsy, assaying the sample by one or more colorimetric methods to determine the level of expression of, and comparing the expression level of SFRP1 in the pre-malignant lesion with the level of expression of in normal breast tissue (i.e., breast tissue that is free of AH).

The present gene and protein expression profiles further may include determining the expression levels of reference or control genes and the proteins. The currently reference genes are ACTB, GAPD, GUSB, RPLP0 and TFRC.

Table 1 identifies the genes in the present gene expression profiles. Table 1 also indicates whether expression of the gene and protein is up- or down-regulated in patients likely to experience progression of a pre-malignant lesion to breast cancer. Table 1 includes the NCBI Accession No. of the reference sequence of each gene (which sequences are incorporated herein by reference); other variants of these genes and proteins exist, which can be readily ascertained by reference to an appropriate database such as NCBI Entrez (available via the NIH website).

TABLE 1

| | NCBI Reference | Up-Regulated (+) or |
|---|---|---|
| Gene | Sequence No. | Down-regulated (−) |
| TP53RK | NM_033550 | + |
| GLUL | NM_002065 | + |
| MKNK2 | NM_199054 | + |
| KDM4B | NM_015015 | + |
| NAPA | NM_003827 | + |
| TTC39A | NM_001144832 | + |
| GREB1 | NM_014668 | + |
| POTEE | NM_001083538 | + |
| POTEM | NM_001145442 | + |
| TMEM25 | NM_001144034 | + |
| DNALI1 | NM_003462 | + |
| MLPH | NM_024101 | + |
| FOXA1 | NM_004496 | + |
| PREX1 | NM_020820 | + |
| KIAA1244 | NM_020340 | + |
| AR | NM_000044 | + |
| CACNA1D | NM_000720 | + |
| ESR1 | NM_000125 | + |
| LRIG1 | NM_015541 | + |
| CRYAB | NM_001885 | − |
| MAML2 | NM_032427 | − |
| DMD | NM_000109 | − |
| TFAP2C | NM_003222 | − |
| SFRP1 | NM_003012 | − |
| NFIB | NM_005596 | − |
| ARRDC3 | NM_020801 | − |
| ANXA1 | NM_000700 | − |
| ANKRD36 | NM_001164315 | − |
| CXCL2 | NM_002089 | |
| SLP1 | NM_032872.2; | |
| | NM_032872.2 | |
| MSX2 | NM_002449 | |
| PRKAR2B | NM_002736 | |
| SGK1 and/or | AJ000512 | |
| SKG3 | | |
| TGFBR3 | NM_001195683.1; | |
| | NM_003243.4; | |
| | NM_001195684.1 | |
| NDRG2 | NM_001282216.1; | |
| | NM_001282215.1; | |
| | NM_001282214.1; | |
| | NM_001282212.1 | |
| CCL28 | NM_001301875.1; | |
| | NM_001301875.1; | |
| | NM_001301874.1; | |
| | NM_148672.3 | |
| KIT1 | | |
| TANK | NM_001199135.1; | |
| | NM_133484.1; | |
| | NM_004180.2 | |

### Assays

The present invention further comprises assays for determining the gene and/or protein expression profile in a patient's sample, and instructions for using the assay. The assay may be based on detection of nucleic acids (e.g., using nucleic acid probes specific for the nucleic acids of interest) or proteins or peptides (e.g., using nucleic acid probes or antibodies specific for the proteins/peptides of interest). In one embodiment, the assays comprises an immunohistochemistry (IHC) test in which tissue samples, such as arrayed in a tissue microarray (TMA), and are contacted with antibodies specific for the proteins/peptides identified in the gene or protein expression profile as being indicative of the likelihood of that a pre-malignant lesion will progress to breast cancer.

The present invention provides methods of detecting target nucleic acids via in situ hybridization and fluorescent in situ hybridization using nucleic acid probes. The methods of in situ hybridization were first developed in 1969 and many improvements have been made since. The basic technique utilizes hybridization kinetics for RNA and/or DNA via hydrogen bonding. By labeling sequences of DNA or RNA of sufficient length (approximately 50-300 base pairs), selective probes can be made to detect particular sequences of DNA or RNA. The application of these probes to tissue sections allows DNA or RNA to be localized within tissue regions and cell types. Methods of probe design are known to those of skill in the art. Detection of hybridized probe and target may be performed in several ways known in the art. Most prominently is through the use of detection labels attached to the probes. Probes of the present invention may be single or double stranded and may be DNA, RNA, or mixtures of DNA and RNA. They may also constitute any nucleic acid based construct. Labels for the probes of the present invention may be radioactive or non-radioactive and the design and use of such labels is well known in the art.

The present invention provides for new assays useful in the diagnosis, prognosis and prediction of pre-malignant breast lesions, and the likelihood that such lesions will progress to breast cancer. The immunoassays of the present invention utilize polyclonal or monoclonal antibodies that specifically bind to proteins expressed from the biomarkers and gene signatures of the present invention in a biological sample. Any type of immunoassay format may be used, including, without limitation, enzyme immunoassays (EIA, ELISA), radioimmunoassay (RIA), fluoroimmunoassay (FIA), chemiluminescent immunoassay (CLIA), counting immunoassay (CIA), immunohistochemistry (IHC), agglutination, nephelometry, turbidimetry or Western Blot. These and other types of immunoassays are well-known and are described in the literature, for example, in Immunochemistry, Van Oss and Van Regenmortel (Eds), CRC Press, 1994; The Immunoassay Handbook, D. Wild (Ed.), Elsevier Ltd., 2005; and the references disclosed therein.

### Kits

The materials for use in the methods of the present invention are suited for preparation of kits produced in accordance with well known procedures. The invention thus provides kits comprising agents, which may include gene-specific or gene-selective probes and/or primers, for quantitating the expression of the disclosed genes for predicting prognostic outcome or response to treatment. Such kits may optionally contain reagents for the extraction of RNA from tumor samples, in particular fixed paraffin-embedded tissue samples and/or reagents for RNA amplification. In addition, the kits may optionally comprise the reagent(s) with an identifying description or label or instructions relating to their use in the methods of the present invention. The kits may comprise containers (including microtiter plates suitable for use in an automated implementation of the method), each with one or more of the various reagents (typically in concentrated form) utilized in the methods, including, for example, pre-fabricated microarrays, buffers, and the like.

The assay methods provided by the present invention may also be automated in whole or in part.

The invention is further illustrated by the following non-limiting examples.

### EXAMPLES

### Example 1

The pre-clinical study was designed to show the diagnostic superiority of the present biomarkers and gene expression profiles in determining the risk that a pre-malignant lesion will progress to breast cancer. In particular it was designed to accurately determine biomarkers and/or gene signatures associated with pre-malignant lesions that progressed to breast cancer.

The study consisted of 22 patients diagnosed as having pre-malignant breast lesions who progressed to invasive cancer subsequent to the diagnosis of AH. The cohort included 12 patients diagnosed with ductal AH, 8 patients diagnosed with lobular AH and/or LCIS (lobular carcinoma in-situ), and 2 patients diagnosed with FEA (flat epithelial atypia).

Formalin-fixed, paraffin embedded diseased (lesion) tissue and matched normal (undiseased) tissue from each patient were analyzed for gene expression using the Affymetrix 1.0 ST chip according to the manufacturer's instructions. Gene expression from the lesions was compared with expression from the normal tissue.

Selected genes that showed significant differences in expression between normal benign tissue and the AH do define the set of genes differentially expressed within each patient. The differentially expressed genes were clustered to define clades and resulted in the 3 patterns designated normal, intermediate and atypical hyperplasia.

The results of the gene expression analysis was analyzed using multivariate analysis relative and other prognostic marker analyses determined for the other cases included in the study. Multivariate analysis was done using Prediction Analysis for Microarrays” (PAM), which performs sample classification from gene expression data, as described by Tibshirani et al., “Diagnosis of multiple cancer types by shrunken centroids of gene expression”, PNAS, (2002) 99:6567-6572 (May 14). The goal of this statistical analysis was to identify biomarkers and/or a gene signature that is predictive of whether a pre-malignant lesion will progress to breast cancer.

The analyses identified approximately 532 genes that are differentially expressed in a statistically meaningful way in pre-malignant lesions that progressed to breast cancer. Of these, a subset of twenty-eight genes was identified that are differentially expressed (i.e., from about 0.5-fold to about 3-fold over- or under-expression compared to normal breast tissue in pre-malignant lesions that progressed to breast cancer. These 28 genes are listed in Table 1, and shown in FIG. 1.

The results identified a further subset of eight genes that are strongly differentially expressed in pre-malignant lesions that progressed to breast cancer: TP53RK, KDM4B, GREB1, FOXA1, ESR1, MAML2, SFRP1 and ANXA1. The level of TP53RK, KDM4B, GREB1, FOXA1 and/or ESR1 are up-regulated, and/or the level of MAML2, SFRP1 and/or ANXA1 are down-regulated in the pre-malignant lesions compared to their expression levels in the normal breast tissue.

The results further showed that one gene, SFRP1, is a strong indicator of the presence of AH in a lesion. SFRP1 is down-regulated in lesions in which AH is present compared to its expression levels in the normal breast tissue, suggesting that this gene is a promising target for therapeutic intervention in patients having pre-malignant lesions in which SFRP1 is down-regulated.

### Example 2

Cell-based data show that CXCL2, SLP1, MSX2, PRKAR2B, and/or SGK are regulated by loss of SFRP1 expression as is found in atypical hyperplasias (AH).

Additionally, data show that TGFBR3, NDRG2, CCL28, KIT1, SLP1, and/or TANK are also regulated by SFRP1 in cells.

Confirmation of the SFRP1/Wnt signaling: The 19 genes differentially expressed between AH and B9 tissue were tested: MAML2, TGFBR3, NDRG2, CCL28, PROM1, NFKBIZ, KIT1, CHI3L1, MUC1, ARRDC3, CXCL2, SLP1, GABRP, TANK, ANXA1, CRYAB, MSX2, PRKAR2B and SGK. Of these, 7 were confirmed to be regulated by Sfrp1 in the same direction as found in AH vs B9: MAML2, ARRDC3, CXCL2, SLP1, MSX2, PRKAR2B, SGK. These 7 genes provide a signature for loss of SFRP1 function.

### BIBLIOGRAPHY

1. Fisher B, Costantino J P, Wickerham D L, Redmond C K, Kavanah M, Cronin W M, Vogel V, Robidoux A, Dimitrov N, Atkins J, Daly M, Wieand S, Tan-Chiu E, Ford L, Wolmark N, (1998), Tamoxifen for prevention of breast cancer: report of the National Surgical Adjuvant Breast and Bowel Project P-1 Study, J Natl Cancer Inst., 90:1371-1388.
2. Bleyer A, Welch H G, (2012), Effect of three decades of screening mammography on breast-cancer incidence. N Engl J Med, 367:1998-2005.
3. Blackburn A C, Hill L Z, Roberts A L, Wang J, Aud D, Jung J, Nikolcheva T, Allard J, Peltz G, Otis C N, Cao Q J, Ricketts R S, Naber S P, Mollenhauer J, Poustka A, Malamud D, Jerry D J, (2007) Genetic mapping in mice identifies DMBT1 as a candidate modifier of mammary tumors and breast cancer risk. Am J Pathol, 170:2030-2041.
4. Jain R K, Mehta R, Dimitrov R, Larsson L G, Musto P M, Hodges K B, Ulbright T M, Hattab E M, Agaram N, Idrees M T, Badve S, (2011) Atypical ductal hyperplasia: interobserver and intraobserver variability. Mod Pathol., 24:917-923.
5. Hartmann L C, Sellers T A, Frost M H, Lingle W L, Degnim A C, Ghosh K, Vierkant R A, Maloney S D, Pankratz V S, Hillman D W, Suman V J, Johnson J, Blake C, Tlsty T, Vachon C M, Melton L J, III, Visscher D W, (2005) Benign breast disease and the risk of breast cancer. N Engl J Med., 353:229-237.
6. Keimling M, Deniz M, Varga D, Stahl A, Schrezenmeier H, Kreienberg R, Hoffmann I, Konig J, Wiesmuller L, (2012) The power of DNA double-strand break (DSB) repair testing to predict breast cancer susceptibility. FASEB J., 26:2094-2104.
7. Koch J G, Gu X, Han Y, El-Naggar A K, Olson M V, Medina D, Jerry D J, Blackburn A C, Peltz G, Amos C I, Lozano G, (2007), Mammary tumor modifiers in BALB/cJ mice heterozygous for p 53. Mamm Genome, 18:300-309.

The invention is described with reference to various specific embodiments and techniques. However, it should be understood that many variations and modifications may be made while remaining within its scope. All referenced publications, patents and patent documents are intended to be incorporated by reference, as though individually incorporated by reference.

What is claimed is:

1. A method of determining the risk that a pre-malignant breast lesion is likely to progress to breast cancer in a patient diagnosed with the pre-malignant lesion, the method comprising detecting the expression level of at least one of TP53RK, GLUL, MKNK2, KDM4B, NAPA, TTC39A, GREB1, POTEE, POTEM, TMEM25, DNALI1, MLPH, FOXA1, PREX1, KIAA1244, AR, CACNA1D, ESR1, LRIG1, CRYAB, MAML2, DMD, TFAP2C, SFRP1, NFIB, ARRDC3, ANXA1, CXCL2, SLP1, MSX2, PRKAR2B, SGK and ANKRD36, and correlating differential expression levels of said genes with an increased risk of progression to breast cancer.

2. The method of claim 1, wherein the method comprises detecting the expression level of at least one of TP53RK, KDM4B, GREB1, FOXA1, ESR1, MAML2, SFRP1 and ANXA1, wherein over-expression of TP53RK, KDM4B, GREB1, FOXA1 and ESR1 or under-expression of MAML2, SFRP1 and ANXA1 are indicative of an increased risk of progression to breast cancer.

3. A method for determining the presence of atypical hyperplasia in a biological sample, comprising detecting the expression level of SFRP1, wherein under-expression of SFRP1 is indicative of the presence of AH.

4. A method for determining the risk that a pre-malignant breast lesion is likely to progress to breast cancer in a human subject diagnosed with the pre-malignant lesion, wherein the pre-malignant breast lesion likely to progress to breast cancer is characterized by differential expression of least one biomarker of TP53RK, GLUL, MKNK2, KDM4B, NAPA, TTC39A, GREB1, POTEE, POTEM, TMEM25, DNALI1, MLPH, FOXA1, PREX1, KIAA1244, AR, CACNA1D, ESR1, LRIG1, CRYAB, MAML2, DMD, TFAP2C, SFRP1, NFIB, ARRDC3, ANXA1, CXCL2, SLP1, MSX2, PRKAR2B, SGK and ANKRD36 comprising:
i) obtaining a biological sample from the subject;
ii) applying an antibody specific for at least one of TP53RK, GLUL, MKNK2, KDM4B, NAPA, TTC39A, GREB1, POTEE, POTEM, TMEM25, DNALI1, MLPH, FOXA1, PREX1, KIAA1244, AR, CACNA1D, ESR1, LRIG1, CRYAB, MAML2, DMD, TFAP2C, SFRP1, NFIB, ARRDC3, ANXA1, CXCL2, SLP1, MSX2, PRKAR2B, SGK and ANKRD36 to the sample, wherein presence of the biomarker creates an antibody-biomarker complex;
iii) detecting and quantifying said complex; and
iv) diagnosing an increased risk of progression to breast cancer by correlating levels of said complex of step iii) with an increased risk of progression to breast cancer.

5. A method for determining the risk that a pre-malignant breast lesion is likely to progress to breast cancer in a human subject diagnosed with the pre-malignant lesion, wherein the pre-malignant breast lesion likely to progress to breast cancer is characterized by differential expression of least one biomarker of TP53RK, GLUL, MKNK2, KDM4B, NAPA, TTC39A, GREB1, POTEE, POTEM, TMEM25, DNALI1, MLPH, FOXA1, PREX1, KIAA1244, AR, CACNA1D, ESR1, LRIG1, CRYAB, MAML2, DMD, TFAP2C, SFRP1, NFIB, ARRDC3, ANXA1, CXCL2, SLP1, MSX2, PRKAR2B, SGK and ANKRD36 comprising:
i) obtaining a biological sample from the subject;
ii) applying a nucleic acid probe specific for at least one of TP53RK, GLUL, MKNK2, KDM4B, NAPA, TTC39A, GREB1, POTEE, POTEM, TMEM25, DNALI1, MLPH, FOXA1, PREX1, KIAA1244, AR, CACNA1D, ESR1, LRIG1, CRYAB, MAML2, DMD, TFAP2C, SFRP1, NFIB, ARRDC3, ANXA1, CXCL2, SLP1, MSX2, PRKAR2B, SGK and ANKRD36 to the sample, wherein presence of the biomarker creates an probe-biomarker complex;
iii) detecting and quantifying said complex; and
iv) diagnosing an increased risk of progression to breast cancer by correlating levels of said complex of step iii) with an increased risk of progression to breast cancer.

6. The method of claim 4 or 5, wherein the method comprises detecting a complex with at least one of TP53RK, KDM4B, GREB1, FOXA1, ESR1, MAML2, SFRP1 and ANXA1, wherein an increase complex of TP53RK, KDM4B, GREB1, FOXA1 and ESR1 or a decreased complex of MAML2, SFRP1 and ANXA1 are indicative of an increased risk of progression to breast cancer.

7. A method for diagnosing atypical hyperplasia in a biological sample from a human subject, wherein the atypical hyperplasia is characterized by the under-expression of SFRP1 biomarker comprising:
i) obtaining a biological sample from the subject;
ii) applying an antibody specific for SFRP1 biomarker to the sample, wherein presence of the biomarker creates an antibody-biomarker complex;
iii) detection and quantifying said complex; and
iv) diagnosing atypical hyperplasia where the complex of step iii) is decreased.

8. A method for diagnosing atypical hyperplasia in a biological sample from a human subject, wherein the atypical hyperplasia is characterized by the under-expression of SFRP1 biomarker comprising:
i) obtaining a biological sample from the subject;
ii) applying a nucleic acid probe specific for SFRP1 biomarker to the sample, wherein presence of the biomarker creates a probe-biomarker complex;
iii) detection and quantifying said complex; and
iv) diagnosing atypical hyperplasia where the complex of step iii) is decreased.

9. A method to treat pre-malignant breast lesion likely to progress to breast cancer in a patient comprising: obtaining the results of an analysis that determined the expression level of at least one of TP53RK, KDM4B, GREB1, FOXA1, ESR1, MAML2, SFRP1 and ANXA1 in a biological sample from the patient and administering treatment to the patient if the patient over-expresses TP53RK, KDM4B, GREB1, FOXA1 and ESR1 or under-expresses MAML2, SFRP1 and ANXA1, so as to inhibit progression to breast cancer.

10. A method for monitoring the progression or effect of treatment of a pre-malignant breast lesion in a subject, said method comprising detecting the expression level of at least one of TP53RK, GLUL, MKNK2, KDM4B, NAPA, TTC39A, GREB1, POTEE, POTEM, TMEM25, DNALI1, MLPH, FOXA1, PREX1, KIAA1244, AR, CACNA1D, ESR1, LRIG1, CRYAB, MAML2, DMD, TFAP2C, SFRP1, NFIB, ARRDC3, ANXA1, CXCL2, SLP1, MSX2, PRKAR2B, SGK and ANKRD36 in a sample from said subject, comparing the level of at least one of TP53RK, GLUL, MKNK2, KDM4B, NAPA, TTC39A, GREB1, POTEE, POTEM, TMEM25, DNALI1, MLPH, FOXA1, PREX1, KIAA1244, AR, CACNA1D, ESR1, LRIG1, CRYAB, MAML2, DMD, TFAP2C, SFRP1, NFIB, ARRDC3, ANXA1, CXCL2, SLP1, MSX2, PRKAR2B, SGK and ANKRD36 in said subject with a standard or with a previous level of at least one of TP53RK, GLUL, MKNK2, KDM4B, NAPA, TTC39A, GREB1, POTEE, POTEM, TMEM25, DNALI1, MLPH, FOXA1, PREX1, KIAA1244, AR, CACNA1D, ESR1, LRIG1, CRYAB, MAML2, DMD, TFAP2C, SFRP1, NFIB, ARRDC3, ANXA1, CXCL2, SLP1, MSX2, PRKAR2B, SGK and ANKRD36 in said subject, wherein a change in the level in said subject correlates with the progression or effect of treatment of the pre-malignant breast lesion in the subject.
